(19) United States
(12) Patent Application Publication
(10) Pub. No.: US 2025/0268745 A1
(43) Pub. Date: Aug. 28, 2025

(54) MOBILE CRYOTHERAPY DEVICE

(71) Applicant: Michael Dent, Towson, MD (US)

(72) Inventor: Michael Dent, Towson, MD (US)

(21) Appl. No.: 18/586,628

(22) Filed: Feb. 26, 2024

(51) Int. Cl. A61F 7/00; B60P 3/00; B60R 13/08

(52) U.S. Cl. CPC A61F 7/0053; A61F 7/0085; B60P 3/00; B60R 13/0823; A61F 2007/0056

(57) ABSTRACT

A mobile cryotherapy device is provided. The device is comprised of a vehicle comprised of an interior space having at least one cooling element. The cooling element can be used to cool the interior space to temperatures required during cryotherapy treatments. In one embodiment, the interior space is comprised of an interior divider such as a flap or a door that allows an exterior door of the vehicle to remain open while keeping the temperature of the interior space constant.

### FIELD OF THE INVENTION

The present invention relates generally to the field of cryotherapy. More specifically, the present invention relates to a mobile cryotherapy device comprised of a vehicle with an interior space comprised of a cooling element that can be used for cryotherapy. Accordingly, the present disclosure makes specific reference thereto. Nonetheless, it is to be appreciated that aspects of the present invention are also equally applicable to other like applications, devices, and methods of manufacture.

### BACKGROUND

Cryotherapy, commonly known as cold therapy, involves exposing the body to extremely cold temperatures for a short period. This technique can be administered in various forms, such as ice packs, coolant sprays, ice massage, and ice baths. The more advanced approach, known as whole-body cryotherapy (WBC), involves stepping into a cryogenic chamber or a cryosauna for a few minutes, where temperatures can plummet as low as −110° C. (−166° F.).

The benefits of cryotherapy are diverse and significant. It is highly effective for pain relief and muscle healing, as the cold exposure can reduce muscle pain and speed up the healing process from muscle damage. This is achieved by temporarily reducing nerve activity. Cryotherapy also plays a crucial role in reducing inflammation, which is particularly beneficial since chronic inflammation is linked to various health issues, including cancer, diabetes, and chronic pain. While cryotherapy alone is not a weight loss solution, it can complement weight loss efforts. The underlying idea is that the body burns more calories while working harder to stay warm in the cold environment. Additionally, there are indications that cryotherapy can enhance mental health and mood, potentially offering relief for conditions like depression and anxiety.

However, a significant limitation of current cryotherapy methods is their lack of portability. Most advanced cryotherapy treatments, especially those involving cryogenic chambers or cryosaunas, require specialized equipment and facilities. These chambers are not only expensive but also large and stationary, making them inaccessible for home use or on-the-go therapy. This limitation means that individuals seeking regular cryotherapy treatments often have to visit specialized centers or clinics, which can be inconvenient and costly. While portable options like ice packs or coolant sprays exist, they do not offer the same level of effectiveness or the controlled environment provided by professional cryotherapy chambers. As a result, the accessibility and convenience of cryotherapy are significantly limited by the current methods and technologies available.

Therefore, there exists a long-felt need in the art for an improved cryotherapy device. There also exists a long-felt need in the art for a mobile cryotherapy device. More specifically, there exists a long-felt need in the art for a mobile cryotherapy device that provides a cryotherapy treatment, wherein the device is easily mobile.

The subject matter disclosed and claimed herein, in one embodiment thereof, comprises a mobile cryotherapy device. The device is comprised of a vehicle comprised of an interior space having at least one cooling element. The cooling element can be used to cool the interior space to temperatures required during cryotherapy treatments. In one embodiment, the interior space is comprised of an interior divider such as a flap or a door that allows an exterior door of the vehicle to remain open while keeping the temperature of the interior space constant.

In this manner, the mobile cryotherapy device of the present invention accomplishes all the foregoing objectives and provides an improved cryotherapy device. More specifically, the device provides a cryotherapy treatment that is easily mobile.

### SUMMARY

The following presents a simplified summary to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a mobile cryotherapy device comprised of a vehicle with an interior space with at least one cooling element that can be used for cryotherapy. The interior space is separated from an exterior door of the vehicle via at least one interior divider.

The vehicle may be any vehicle type but is preferably a box truck, a semi-truck and trailer, a cargo van, or any other vehicle with at least one rear exterior door and a large interior that can accommodate multiple individuals. The interior space of the vehicle is comprised of at least one cooling element capable of cooling the interior space to between −25° F. to 32° F. during cryotherapy. The interior space of the vehicle is separated from the outside via at least one exterior door.

The interior space is also comprised of at least one insulating interior divider that allows the interior space to retain temperature while the exterior door is open. In one embodiment, the divider is comprised of at least one curtain/flap (preferably vinyl) that may be motorized or manually moveable to allow users to easily walk into the cooled portion of the interior space, wherein the divider also retains the temperature within said portion. In another embodiment, the divider may be comprised of an immovable insulated wall or partition comprised of at least one insulated door that allows a user to access the cooled area of the interior space.

The present invention is also comprised of a method of using the device. First, a device is provided comprised of a vehicle comprised of at least one exterior door and an interior space comprised of at least one cooling element and at least one interior divider. Then, a user can activate the cooling element to cool the interior space between −25° F. to 32° F. . . . Next, a user can open the exterior door and enter the interior space through the divider, which is preferably a flap or curtain. Finally, a user can remain in the interior space for a period of time during a cryotherapy session.

The present invention is also comprised of a second method of using the device. First, a device is provided comprised of a vehicle comprised of at least one exterior door and an interior space comprised of at least one cooling element, and at least one interior divider comprised of at least one door. Then, a user can activate the cooling element to cool the interior space between-25° F. to 32° F. . . . Next, a user can open the exterior door and enter the interior space through the door of the divider, which is preferably a wall. Finally, a user can close the door and remain in the interior space for a period of time during a cryotherapy session.

Accordingly, the mobile cryotherapy device of the present invention is particularly advantageous as it provides an improved cryotherapy device. More specifically, the device provides a cryotherapy treatment that is easily mobile. In this manner, the mobile cryotherapy device overcomes the limitations of existing cryotherapy devices known in the art.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a rear perspective view of one potential embodiment of a mobile cryotherapy device of the present invention in accordance with the disclosed architecture;

FIG. 2 illustrates a rear perspective view of one potential embodiment of a mobile cryotherapy device of the present invention with an interior divider in accordance with the disclosed architecture;

FIG. 3 illustrates a rear perspective view of one potential embodiment of a mobile cryotherapy device of the present invention with an interior divider in accordance with the disclosed architecture;

FIG. 4 illustrates a flowchart of a method of using one potential embodiment of a mobile cryotherapy device of the present invention in accordance with the disclosed architecture; and

FIG. 5 illustrates a flowchart of a method of using one potential embodiment of a mobile cryotherapy device of the present invention in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth to provide a thorough understanding thereof. It may be evident, however, that the innovation can be practiced without these specific details. In other instances, well-known structures and devices are shown in block diagram form to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt need in the art for an improved cryotherapy device. There also exists a long-felt need in the art for a mobile cryotherapy device. More specifically, there exists a long-felt need in the art for a mobile cryotherapy device that provides a cryotherapy treatment, wherein the device is easily mobile.

The present invention, in one exemplary embodiment, is comprised of a mobile cryotherapy device. The device is comprised of a vehicle with an interior space with at least one cooling element. The cooling element can be used to cool the interior space to cryotherapy temperatures. The interior space is separated from an exterior door of the vehicle via at least one interior divider.

The vehicle may be any vehicle type but is preferably a box truck, a semi-truck and trailer, a cargo van, or any other vehicle with at least one rear exterior door and a large interior that can accommodate multiple individuals. The interior space of the vehicle is comprised of at least one cooling element capable of cooling the interior space to between −25° F. to 32° F. during cryotherapy. The interior space of the vehicle is separated from the outside via at least one exterior door.

The interior space is also comprised of at least one insulating interior divider. The divider allows the interior space to retain temperature while the exterior door is open. In one embodiment, the divider is comprised of at least one curtain/flap (preferably vinyl) that may be motorized or manually moveable to allow users to easily walk into the cooled portion of the interior space, wherein the divider also retains the temperature within said portion. In another embodiment, the divider may be comprised of an immovable insulated wall or partition comprised of at least one insulated door that allows a user to access the cooled area of the interior space.

The present invention is also comprised of a method of using the device. First, a device is provided comprised of a vehicle comprised of at least one exterior door and an interior space comprised of at least one cooling element and at least one interior divider. Then, a user can activate the cooling element to cool the interior space between −25° F. to 32° F. . . . Next, a user can open the exterior door and enter the interior space through the divider, which is preferably a flap or curtain. Finally, a user can remain in the interior space for a period of time during a cryotherapy session.

The present invention is also comprised of a second method of using the device. First, a device is provided comprised of a vehicle comprised of at least one exterior door and an interior space comprised of at least one cooling element, and at least one interior divider comprised of at least one door. Then, a user can activate the cooling element to cool the interior space between-25° F. to 32° F. . . . Next, a user can open the exterior door and enter the interior space through the door of the divider, which is preferably a wall. Finally, a user can close the door and remain in the interior space for a period of time during a cryotherapy session.

Accordingly, the mobile cryotherapy device of the present invention is particularly advantageous as it provides an improved cryotherapy device. More specifically, the device provides a cryotherapy treatment that is easily mobile. In this manner, the mobile cryotherapy device overcomes the limitations of existing cryotherapy devices known in the art.

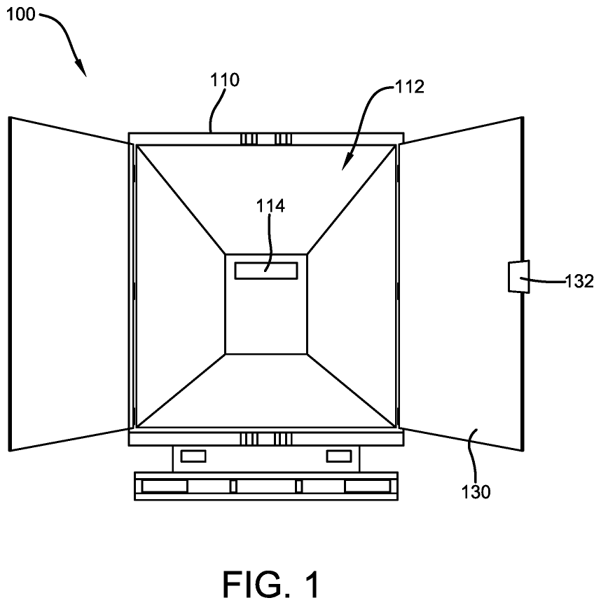

Referring initially to the drawings, FIG. 1 illustrates a rear perspective view of one potential embodiment of a mobile cryotherapy device 100 of the present invention in accordance with the disclosed architecture. The device 100 is comprised of a vehicle 110 with an interior space 112 with at least one cooling element 114 that can be used for cryotherapy. The interior space 112 may be an insulated interior space in one embodiment. The interior space 112 is separated from an exterior door 130 of the vehicle 110 via at least one interior divider 120. The vehicle 110 may be any vehicle type known in the art. However, the vehicle 110 is preferably a box truck, a semi-truck and trailer, a cargo van, or any other vehicle with at least one rear exterior door 130 and a large interior that can accommodate multiple individuals.

The interior space 112 of the vehicle 110 is comprised of at least one cooling element 114. The cooling element 114 may be any cooling element known in the art capable of cooling the interior space 112 to between −25° F. to 32° F. during cryotherapy. In one embodiment, the cooling element 114 is comprised of liquid nitrogen or an electric cooling element. However, it should be appreciated that the element 114 may cool the interior space 112 within temperature range.

The interior space 112 of the vehicle 110 is separated from the outside via at least one exterior door 130. The door 130 may be any style such as, but not limited to, a roll-up door, a hinged door, etc. The door 130 may be secured via at least one locking mechanism 132 of any type known in the art to prevent inadvertent opening of the door 130.

The interior space 112 is also comprised of at least one insulating interior divider 120. The divider 120 allows the interior space 112 to retain consistent temperature while the exterior door 130 is open. The divider 120 may be a plurality of different structures in different embodiments.

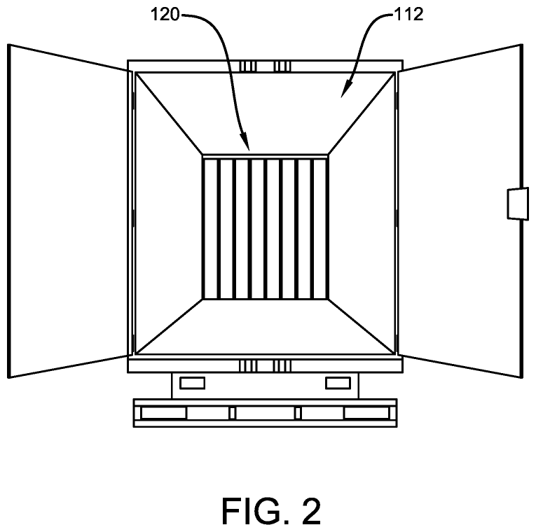

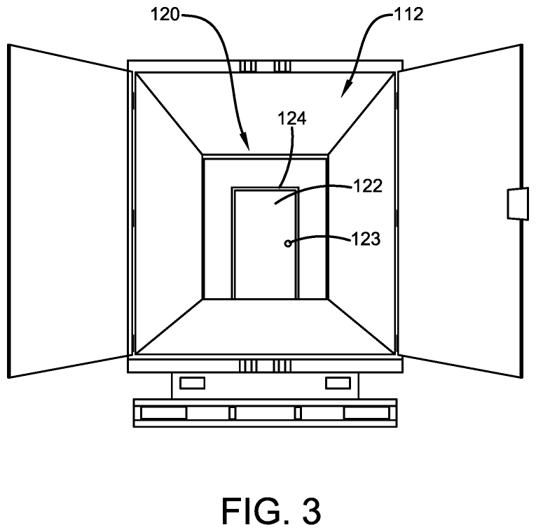

In one embodiment, the divider 120 is comprised of at least one curtain/flap as seen in FIG. 2 (preferably vinyl) that may be motorized or manually moveable to allow users to easily walk into the cooled portion of the interior space 112, wherein the divider 120 also retains the temperature within said portion. In another embodiment, the divider 120 may be comprised of an immovable insulated wall or partition. In this embodiment, the divider 120 is comprised of at least one insulated door 122 that allows a user to access the cooled area of the interior space 112, as seen in FIG. 3. The door 122 may be comprised of a locking mechanism 123 of any type that keeps the door 122 closed during use. The door 122 may be comprised of at least one seal 124 that prevents air from escaping around the door.

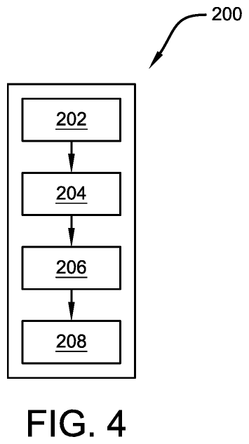

The present invention is also comprised of a method of using 200 the device 100, as seen in FIG. 4. First, a device 100 is provided comprised of a vehicle 110 comprised of at least one exterior door 130 and an interior space 112 comprised of at least one cooling element 114 and at least one interior divider 120 [Step 202]. Then, a user can activate the cooling element 114 to cool the interior space 112 between −25° F. to 32° F. [Step 204]. Next, a user can open the exterior door 130 and enter the interior space 112 through the divider 120, which is preferably a flap or curtain [Step 206]. Finally, a user can remain in the interior space 112 for a period of time during a cryotherapy session [Step 208].

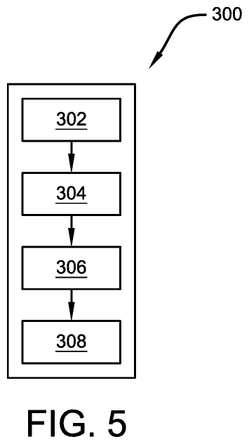

The present invention is also comprised of a second method of using 300 the device 100, as seen in FIG. 5. First, a device 100 is provided comprised of a vehicle 110 comprised of at least one exterior door 130 and an interior space 112 comprised of at least one cooling element 114 and at least one interior divider 120 comprised of at least one door 122 [Step 302]. Then, a user can activate the cooling element 114 to cool the interior space 112 between −25° F. to 32° F. [Step 304]. Next, a user can open the exterior door 130 and enter the interior space 112 through the door 122 of the divider 120, which is preferably a wall [Step 306]. Finally, a user can close the door 122 and remain in the interior space 112 for a period of time during a cryotherapy session [Step 308].

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “mobile cryotherapy device” and “device” are interchangeable and refer to the mobile cryotherapy device 100 of the present invention.

Notwithstanding the foregoing, the mobile cryotherapy device 100 of the present invention and its various components can be of any suitable size and configuration as is known in the art without affecting the overall concept of the invention, provided that they accomplish the above-stated objectives. One of ordinary skill in the art will appreciate that the size, configuration, and material of the mobile cryotherapy device 100 as shown in the FIGS. are for illustrative purposes only, and that many other sizes and shapes of the mobile cryotherapy device 100 are well within the scope of the present disclosure. Although the dimensions of the mobile cryotherapy device 100 are important design parameters for user convenience, the mobile cryotherapy device 100 may be of any size, shape, and/or configuration that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A mobile cryotherapy device comprising:
a vehicle comprised of an interior space and an exterior door; and
a cooling element positioned within the interior space.

2. The mobile cryotherapy device of claim 1, wherein the vehicle is comprised of a box truck, a semi-truck and a trailer, or a cargo van.

3. The mobile cryotherapy device of claim 1, wherein the cooling element can cool the interior space between −25° F. to 32° F.

4. The mobile cryotherapy device of claim 1, wherein the cooling element is comprised of a liquid nitrogen cooling element.

5. A mobile cryotherapy device comprising:
a vehicle comprised of an interior space and an exterior door;
a cooling element positioned within the interior space; and
an interior divider positioned within the interior space.

6. The mobile cryotherapy device of claim 5, wherein the vehicle is comprised of a box truck, a semi-truck and a trailer, or a cargo van.

7. The mobile cryotherapy device of claim 5, wherein the cooling element can cool the interior space between −25° F. to 32° F.

8. The mobile cryotherapy device of claim 5, wherein the cooling element is comprised of a liquid nitrogen cooling element.

9. The mobile cryotherapy device of claim 5, wherein the interior divider is comprised of a flap or a curtain.

10. The mobile cryotherapy device of claim 5, wherein the interior divider is comprised of an insulated wall.

11. The mobile cryotherapy device of claim 10, wherein the interior divider is comprised of an insulated door.

12. The mobile cryotherapy device of claim 11, wherein the insulated door is comprised of a seal.

13. A method of using a mobile cryotherapy device, the method comprising the following steps:
providing a mobile cryotherapy device comprised of a vehicle comprised of an exterior door and an interior space comprised of a cooling element and an interior divider;
activating the cooling element to cool the interior space between −25° F. to 32° F.;
opening the exterior door and entering the interior space through the divider; and
remaining in the interior space for a period of time during a cryotherapy session.

14. The method of using a mobile cryotherapy device of claim 13, wherein the interior divider is comprised of a flap or a curtain.

15. The method of using a mobile cryotherapy device of claim 13, wherein the interior divider is comprised of an insulated wall.

16. The method of using a mobile cryotherapy device of claim 15, wherein the interior divider is comprised of an insulated door.

17. The method of using a mobile cryotherapy device of claim 16, wherein the insulated door is comprised of a seal.

18. The method of using a mobile cryotherapy device of claim 13, wherein the vehicle is comprised of a box truck, a semi-truck and a trailer, or a cargo van.

19. The method of using a mobile cryotherapy device of claim 13, wherein the cooling element is comprised of a liquid nitrogen cooling element.

20. The method of using a mobile cryotherapy device of claim 16, wherein the insulated door is comprised of a locking mechanism.
